(19) United States
(12) Patent Application Publication
(10) Pub. No.: US 2025/0017627 A1
(43) Pub. Date: Jan. 16, 2025

(54) MEDICAL BONE IMPLANT AND METHOD FOR MONITORING THE CONDITION OF AN IMPLANT

(71) Applicant: ICOTEC AG, Altstätten (CH)

(72) Inventors: Albert Maria VODERMAYER, Dietlikon (CH); Ramon Jürgen HÜPPI, Marbach (CH); Sebastian HEIM, Altstätten (CH); Roger Heinrich STADLER, Altstätten (CH); Andreas Christian DÜNKI, Reutlingen (CH)

(21) Appl. No.: 18/714,967

(22) PCT Filed: Dec. 2, 2022
(86) PCT No.: PCT/EP2022/084161

§ 371 Date: May 30, 2024

(30) Foreign Application Priority Data
Dec. 17, 2021 (EP) 21215570.9

(51) Int. Cl. A61B 17/70; A61B 17/00

(52) U.S. Cl. CPC A61B 17/7031; A61B 2017/0011

(57) ABSTRACT

A medical bone implant for stabilizing a bone region is provided, comprising an implant body manufactured from a thermoplastic composite material for the purpose of being anchored in or on a human or animal bone for the purpose of stabilizing the bone region, and further comprising at least one sensor for measuring one or more parameter(s) of the healing process and/or of a condition of the implant. The at least one sensor is a passive, magnetoelastic sensor. Furthermore, a method for monitoring the condition of an implant is provided. A passive magnetoelastic sensor is used for monitoring the condition of the implant during its manufacturing process and/or during its storage.

### CROSS-REFERENCE TO RELATED APPLICATIONS

This application is a national stage application, filed under 35 U.S.C. § 371, of International Patent Application No. PCT/EP2022/084161 filed Dec. 2, 2022, which claims the benefit of European Patent Application No. 21215570.9 filed Dec. 17, 2021, the entire disclosures of which are incorporated herein by reference for all purposes.

### TECHNICAL FIELD

The present disclosure relates to a medical bone implant for stabilizing a bone region with an implant body manufactured from a thermoplastic composite material, such as is often used especially for the bracing of the spine or in the treatment of trauma. Furthermore, the disclosure concerns a method for monitoring the condition of an implant.

### BACKGROUND

In the field of medicine, implants are used increasingly often and for various reasons. An implant is an artificial device which is inserted into the human or animal body for a specific, mostly prolonged time period. The implant can, for example, serve to support or replace a body function, such as is the case for example with cardiac pacemakers, cochlea implants, or prostheses. Implants, however, can also serve to replace a destroyed body part or to enlarge a body part (plastic surgery), or to monitor the user, such as often is the case with RFID chips for pets.

An important group of implants is bone implants, which are often used for bracing the spine or in the treatment of trauma. Bone implants are at least partially, mostly entirely arranged inside the body, and thereby are anchored in or on a bone, that is, attached thereto.

With implants generally, and especially in the case of bone implants, the condition of the implant as well as the healing process of the patient is of interest during the entire application of the implant in the body. The condition of the implant, for example, can be related to deformations of the implant and thus forces impacting the implant, based on which conclusions can be drawn about the healing process. Vice versa, the healing process can influence the condition of the implant and, for example, its life span.

Therefore it is known to provide sensors in test implants, but also in implants inserted into the body for a prolonged period, in order to offer the medical personnel the possibility of receiving information about the healing process.

In US 2017/0196508 A1, the provision of sensors in or on spinal implants is described. The sensors can especially be acceleration meters and/or tensometers, which can be arranged at various points of the pedicle system, in order to monitor the healing process. Among others, the use of passive MEMS-(micro-electro-mechanical system-) sensors is proposed.

Further documents, which are derived from the same applicant or owner, respectively, as the US 2017/0196508 A1, and also disclose the provision of passive MEMS-sensors in implants, are WO 2015/20070 A1, WO 2015/200723 A1, WO 2015/200707 A1, WO 2015/200722 A2, WO 2015/200720 A2, WO 2014/209916 A1, WO 2014/144107 A1, WO 2014/144070 A1, WO 2014/100795 A1 and WO 2015/200718 A1.

MEMS-sensors, however, are relatively elaborate in their manufacturing and require further electronic components for the purpose of wireless reading.

In U.S. Pat. No. 9,629,583 B2, a screw is disclosed which is screwable into a bone, and in the interior of which a sensor, as well as means for processing, storage, and energy storage, are arranged.

WO 2017/004483 A1 discloses a pedicle system with a sensor attached to a connecting rod for the measurement of compression, deflection, and/or torsion of the connecting rod. Likewise, an intervertebral placeholder with a sensor is disclosed. The sensor is a tensometer strip or a piezoelectric sensor. Both types of sensors are relatively elaborate in their manufacturing and accordingly expensive. Furthermore, further electronic components are necessary in order to enable a wireless reading of the sensor data.

WO 2007/090005 A1 concerns spinal implants. The spinal implants are inserted between two neighboring vertebral bodies and comprise a sensor for measuring loads. A tensometer strip is specified as an exemplary sensor.

WO 2007/098385 A2 discloses a sensor, which is in each case implantable between two vertebral bodies, in order to measure the loads occurring there. The reading of the sensor can be carried out in a wireless manner, for example via RFID.

The WO 2007/025191 A1 discloses the attachment of sensors on an orthopedic bone implant, such as especially an intramedullary nail, for the measurement of deflection, torsion, and compression of the implant. The sensors are inserted in indentations, which are provided on the exterior of the implant. The measurement data can be transmitted to the exterior in a wireless manner.

In the patent application WO 2017/116343 A1, a pedicle system is disclosed, which comprises a sensor arranged on the connecting rod, for measuring and transferring force-, deformation- and displacement data.

Furthermore, WO 2021/15485 A2, EP 3 772 350 A1, WO 2020/247890 A1, WO 2017/165717 A1 and U.S. Pat. No. 11,042,916 B1 each disclose implants with active sensors, that is with sensors which are supplied with power for example via a battery mounted in the implant, in order to carry out measurements and to store the measured data in a memory chip which is also arranged in the implant. The reading is then usually carried out via a transmitting unit which is supplied with power by the battery.

The documents WO 2020/206373 A1 and WO 2016/044651 A1 each suggest arranging passive sensors in an implant, however, they do not describe further, on which principle of measurement these sensors shall be based.

The two documents WO 2007/116218 A1 and EP 3 150 998 B1, both from a different technical field, each describe the measurement of mechanical properties of components by means of a micro wire and by exploiting the Barkhausen-effect.

WO 2020/035217 A1 suggests the use of a micro wire in medical devices which are developed for the administration of a medicament, such as for example insulin pumps.

### SUMMARY

The present disclosure provides a medical bone implant which is easy to manufacture, which serves to stabilize a bone region and which comprises an implant body manufactured from a thermoplastic composite material and with at least one sensor, which allows a wireless reading of sensor measurement data. Additionally, the present disclosure provides a simple process for the monitoring of the condition of an implant.

In accordance with an aspect of the present disclosure, a medical bone implant for stabilizing a bone region is provided, which implant comprises:
an implant body manufactured from a thermoplastic composite material, for being anchored in or on a human or animal bone for the purpose of stabilizing the bone region;
as well as at least one sensor for measuring one or more parameters of the healing process and/or of a condition of the implant.

The at least one sensor is a passive magnetoelastic sensor.

By using a passive magnetoelastic sensor, the implant can be manufactured in an especially simple and accordingly inexpensive way. A further advantage is that a passive magnetoelastic sensor usually requires relatively little space in the implant and therefore only minimally influences the implant characteristics. Nevertheless, a passive magnetoelastic sensor optionally allows a wireless reading of the sensor data from outside the patient body. No additional electronic components, such as for example an electric circuit or a separate transmission unit are necessary. Especially, the implant also does not require any energy storage, such as a battery.

As already mentioned, bone implants are at least partially, mostly completely arranged inside the body and thus are anchored in or on a bone, i.e. attached thereto. The anchoring of the implant in or on a bone can therein also be carried out in an indirect manner, i.e. for example via a further artificially manufactured component, especially via a further implant with preferably an implant body also manufactured from a thermoplastic composite material. This is usually the case for example with pedicle systems, where a first bone implant in the form of a connecting rod is anchored via a plurality of second bone implants in the form of pedicle screws in the vertebral bodies.

A bone implant then serves to stabilize a bone region if it supports and/or enforces a bone region. The bone region preferably concerns at least that region of a bone, in or on which the implant body is anchored. Thus, the bone region to be stabilized can concern only one, or alternatively several bones or bone parts. Especially, the bone region can concern a plurality of vertebral bodies of the spine. However, the bone region to be stabilized can for example also concern only a single bone, which for example must be stabilized by means of a bone implant after a fracture, such that the bone parts are allowed to grow together again as desired. A bone implant can also be for example a vertebral body replacement or a spinal disc replacement, as usually these too are anchored on or in at least one bone, i.e. attached thereto. An artificial vertebral body replacement and an artificial spinal disc replacement serve to replace a dorsal vertebra or a spinal disc, respectively, and thus are usually anchored on or in at least one neighboring vertebral body, mostly on or in the two neighboring vertebral bodies. The vertebral body replacement and the spinal disc replacement in each case thus serve to stabilize the spine and especially the bone region with the two neighboring vertebral bodies.

The implant body usually forms the main component of the implant, which preferably gives the implant its structural stability. The implant can especially consist exclusively of implant body and the at least one sensor.

The at least one sensor forms a component which can record specific characteristics, especially physical characteristics of its surroundings in a qualitative manner or as a measured variable in a quantitative manner. The surroundings therein are usually formed by the implant body and/or the immediate region of the human or animal body around the implant body, respectively. Prior to implanting the implant in the human or animal body, respectively, the surroundings however can for example also be formed by a compression molding tool (during the manufacturing process) or by a mounting- and transport arrangement or by other such implants (during the storage or during the transport). The measured variable therein is recorded based on the magnetoelastic effect and is usually transformed into an electric signal during the reading. The implant can comprise a single magnetoelastic sensor or a plurality of magnetoelastic sensors.

The one or more parameters of the healing process and/or of the condition of the implant can especially be a mechanical load, such as for example a compression, a stretching, a deflection or a torsion, and/or a temperature of the implant. The one or more measured parameter(s) can alternatively or additionally also be for example the temperature of the human or animal body, respectively, in the immediate vicinity of the implant. Generally, the sensor can be designed for measuring any parameters which can result in a change of the magnetic characteristics of the sensor material. The one or more parameters thus enable to draw conclusions about the condition of the implant, i.e. information e.g. about pressure, tensile stress, and/or torsional forces which affect the implant, can be obtained. Especially, conclusions about the stability of the implant region, i.e. the region immediately surrounding the implant, can be drawn. The implant region thus can especially concern other implants and/or body structures connected with the implant, such as for example bones. In case of a plurality of measurements, which are carried out at different points in time, the chronological sequence of these parameters and thus any possible changes of the condition of the implant can be evaluated. From the measured parameters or the condition of the implant, respectively, and especially from their course, conclusions can then in turn be drawn about the healing process. The measurement of the temperature for example can allow to draw conclusions about an infection in the immediate vicinity surrounding the implant.

The measurement especially is based on a change of the magnetic characteristics of a material of the sensor. Especially, the load of a force on the implant changes the magnetic permeability of the sensor material. A change of the magnetic permeability of the sensor material can also be the result of a change of temperature. The sensor material therein preferably comprises a pre-magnetization. Due to the magnetoelastic effect, which is also known by the name “Villari-effect”, a load and/or temperature applied to the sensor material results in a change of the magnetic field of the sensor material, which is measurable. The measurement can be carried out especially in a wireless manner, which is especially advantageous, as no-touch measurements are enabled from outside the body.

In order to enable a measurement based on the magnetoelastic effect, the at least one sensor usually comprises at a sensor material in the form of a ferromagnetic material, preferably a ferromagnetic alloy, with an inverted magnetostriction. The ferromagnetic material changes its magnetic characteristics under mechanical or thermal stress, respectively, and especially its magnetic permeability, which can be detected and measured by means of a suitable measuring instrument or reading device, respectively.

It has been found that especially exact measurements are possible when the at least one sensor for measuring the one or more parameter(s) exploits the Barkhausen-effect. Due to the Barkhausen-effect, a continuously changing magnetic field results a discontinuous change of the magnetization of ferromagnetic materials. The reason for this lies in the presence of regions of uniform direction of magnetization, the so-called magnetic domains (german: “Weiss-Bezirke”), which are separated by Bloch-walls. For example, in case of a slow increase of the magnetic field force, the magnetic moments of entire regions, i.e. of the magnetic domains (german: “Weiss-Bezirke”), suddenly are overturned, and thus lead to an abrupt change of the magnetic field of the respective material. Due to the preferred exploitation of the Barkhausen-effect by the at least one sensor, these abrupt magnetic field changes are measurable.

An especially sensitive and exact measurement is possible when the at least one sensor is a micro wire. The usual geometric dimensions of a micro wire can result in an especially strong signal during the measurement due to the magnetoelastic effect. In case of a micro wire, the signal is also especially strong, when the Barkhausen-effect is exploited for the measurement. A micro wire can additionally be especially advantageously arranged in the interior of an implant, for example such that it extends along its main longitudinal center line.

The micro wire preferably has a diameter of 10 μm to 250 μm. The length of the micro wire is preferably more than 25 times, especially preferably more than 50 times, most preferably more than 100 times larger than the diameter.

In a preferred embodiment, the at least one sensor is at least partially, more preferably entirely arranged in the interior of the implant body. The sensor thereby is optimally protected from exterior influences and can deliver measured variables directed from the interior of the implant. Vice versa, however, the surrounding body tissue is separated from the sensor, such that any intolerances with the sensor material can be prevented. In order to achieve optimal results of measurement, the at least one sensor can especially be embedded in the material of the implant body.

In an especially preferred embodiment, the implant body is manufactured from a fibre-reinforced plastic material. The fibre-reinforced plastic material preferably is a carbon fibre reinforced plastic material, such as for example polyetheretherketone (PEEK). The manufacture of the implant body from a thermoplastic plastic material and especially from a fibre-reinforced plastic material on the one hand has the advantage that the manufacture of structurally very stable implants is possible, which additionally are biocompatible and advantageous for imaging processes, such as e.g. MRI. On the other hand, however, thermoplastic plastic material and especially fibre-reinforced plastic material, compared to metals, for example, is also permeable for electromagnetic radiation. Carrying out sensor measurements is therefore easily possible if the sensor is entirely arranged in the interior of the implant body.

In case the implant body is manufactured from a fibre-reinforced plastic material, the fibre length preferably is at least 1 mm, preferably at least 5 mm. Especially preferably, the length of the fibres however is so large that it corresponds to the complete length of the main direction of extension of the implant body. This imparts the implant body with an especially high stiffness and structural stability.

A good implant stability is achieved when the fibre volume content lies in a range of 20-80%, preferably in a range of 35-70%, especially preferably in a range of 45-60%.

As plastic materials, preferably thermoplastics are used, especially preferably so-called high temperature thermoplastics of the families of polyaryletherketones, polyimides and/or polysulfones.

Especially preferred is an embodiment in which the implant comprises a main longitudinal center line, and in which the at least one sensor extends essentially along the entire longitudinal extension of this main longitudinal center line. The main longitudinal center line constitutes a line which extends along its entire longitudinal extension centrally inside the implant, especially inside the implant body. The at least one sensor therefore preferably has essentially the same length as the implant. If the implant describes e.g. an altogether bent or spiral form, the main longitudinal center line also comprises a correspondingly bent or spirally formed design, respectively, such that the at least one sensor also comprises an altogether bent or spirally formed design. This has the advantage that measurements along the entire longitudinal extension of the implant are possible, such that the measurement can encompass the entire implant and is not limited to individual points of the implant. The implant thus can be entirely controlled and monitored over its entire length. a

If the at least one sensor is a micro wire, it can either extend entirely continuously or in pieces as adjoining aligned sections essentially along the entire longitudinal extension of this main longitudinal center line. In other words, the implant can comprise a plurality of sensors, i.e. micro wires, which are arranged in regularly spaced intervals along the main longitudinal center line. In case several aligned sections are provided, they are preferably arranged in a regularly spaced manner to each other. One advantage of several aligned sections can be that separate measurements for the different sections are possible, such that e.g. differences in the load distribution along the main longitudinal center line of the implant body become measurable.

The implant can also contain several such sensors, which extend parallel to each other. Preferably, the sensors extend not only parallel to each other, but also parallel to the main longitudinal center line, which, depending on the design of the implant, can have a bent configuration. By providing several sensors which are arranged parallel to each other, it becomes possible to detect torsional forces which affect the implant.

The implant can especially be a spinal implant, such as an artificial replacement of a vertebral body, an artificial replacement of a spinal disk, a dowel, a screw-anchor, a fixation plate, a pedicle screw, or a connecting rod of a pedicle system. In case of a fixation plate, the fixation plate can be a plate for fixing the spine in an anterior, lateral or posterior region. As an alternative, the implant can also be a trauma implant, such as especially a general anchor system, in other words for example a screw anchor or a dowel, or a bone plate or a bone screw.

The at least one sensor preferably has a temperature stability up to at least 450° C. That means that the at least one sensor is not damaged as long as it is subjected to a temperature which does not exceed 450° C. Advantageously, measurements up to a temperature of 450° C. are also possible by means of the at least one sensor. Such a configuration of the at least one sensor has advantages in terms of the implant manufacturing process. Thereby, the at least one sensor can be integrated in the implant at an early point in time during the manufacturing process, without being damaged by the increased temperature values. Advantageously, it is thereby even possible to carry out measurements by means of the at least one sensor, already during manufacturing process, in order to monitor the manufacturing process. For example, influences affecting the implant body during the manufacturing process, such as especially pressure and temperature, can be measured, in order to draw conclusions about the quality of the implant.

The present disclosure furthermore relates to a method for monitoring a condition of an implant and/or of the healing process of a human or animal body, into which the implant is inserted, wherein the implant especially can be a bone implant as described above, and wherein the implant comprises at least one passive magnetoelastic sensor for measuring one or more parameters of the healing process and/or the condition of the implant. Therein, the sensor is used for monitoring the condition of the implant during its manufacturing process and/or during its storage.

Preferably, the development of pressure and temperature is measured and advantageously also recorded during the manufacturing process and/or during storage of the implant. This way, the integrity and the intactness of the implant can be ensured. Implants, whose pressure and/or temperature for example do not progress within specific margins during the manufacturing process and/or storage, can be discarded. This way, it can be prevented that implants containing manufacturing defects and/or which have been damaged during storage are implanted into the human or animal body. Such a monitoring of the implant by means of the at least one sensor already during the manufacturing process and/or storage is not only possible with bone implants, but also with any other implants.

Preferably, the condition of an implant is not only controlled during the manufacturing process and/or during storage, but also immediately following its implantation into a human or animal body. By “immediately following its implantation” it is meant that e.g. the forces affecting the implant directly after its insertion into the body are measured, thus at the very onset of the healing process. This way, especially the correct positioning of the implant in the body can be verified.

Alternatively, or in addition, but preferably, the healing process following the implantation of the implant into a human or animal body is monitored by means of the at least one sensor. The monitoring, thus e.g. the measurement of the forces affecting the implant and/or the measurement of the temperature by means of the at least a sensor, can especially be carried out in regular time lags. By means of the course of e.g. the forces affecting the implant, conclusions can be drawn about the healing process, and depending thereon, the treatment can for example be terminated or adjusted.

For the manufacturing of the implant, preferably a plurality of prepregs, especially unidirectional prepregs are used, which are pressure-grouted to each other under pressure and heat. Prepregs are understood to be fibre-matrix-semifinished products, thus semifinished products with reinforcement fibres, which are preferably arranged in a plastic matrix. The reinforcement fibres are preferably carbon fibres. The prepregs can e.g. be present in the form of a strand, band or plate. By pressure-grouting the prepregs under pressure and temperature, the prepregs are preferably continuously welded and brought into the final form of the implant. After the pressure-grouting, the prepregs preferably form the implant body.

Prior to the pressure-grouting, the at least one sensor can be arranged between the prepregs, which are then pressure-grouted to each other such that the sensor is also arranged between the prepregs during the pressure-grouting process, and thereby comes to lie at least partially, preferably entirely, in the interior of the implant body in the final implant. Alternatively, the at least one sensor can already be embedded in one of the prepregs during the pressure-grouting process. In that case, the embedding of the sensor in one of the prepregs already takes place prior to the pressure-grouting. In this way, the sensor can be optimally positioned in the implant.

### BRIEF DESCRIPTION OF THE DRAWINGS

Preferred embodiments of the disclosure are described in the following with reference to the drawings, which are merely for the purpose of illustration and not to be interpreted as to be limiting. In the drawings,

FIG. 1 shows a schematic view of a bone implant in the form of a pedicle fixation system and anchored in the vertebral bodies of a spine according to a first embodiment of the disclosure;

FIG. 2a shows a lateral view of a connecting rod of the pedicle fixation system of FIG. 1;

FIG. 2b shows a cross-sectional view through the connecting rod of FIG. 2a in the plane II-II;

FIG. 3a shows a lateral view of a bone implant in the form of a connecting rod of a pedicle fixation system according to a second embodiment of the disclosure;

FIG. 3b shows a cross-sectional view through the connecting rod of FIG. 3a in the plane III-III;

FIG. 4a shows a lateral view of a bone implant in the form of a connecting rod of a pedicle fixation system according to a third embodiment of the disclosure;

FIG. 4b shows a cross-sectional view through the connecting rod of FIG. 4a in the plane IV-IV;

FIG. 5 shows a flow diagram of a process for the manufacture of a bone implant according to a first variant of the disclosure;

FIG. 6 shows a flow diagram of a process for the manufacture of a bone implant according to a second variant of the disclosure;

FIG. 7a shows a schematic cross-sectional view of several prepregs inserted in a pressure-grouting tool, immediately prior to pressure-grouting for the purpose of manufacturing a different bone implant according to the disclosure;

FIG. 7b shows a schematic cross-sectional view of the pressure-grouting tool of FIG. 7a during the pressure-grouting of the prepregs;

FIG. 8 shows a schematic view of the manufacturing step of FIG. 7b during the reading of the sensor for the purpose of monitoring the pressure-grouting process; and

FIG. 9 shows a schematic view of the bone implant of FIG. 1 during the reading of the sensor for the purpose of monitoring the healing process.

### DETAILED DESCRIPTION OF THE EMBODIMENTS

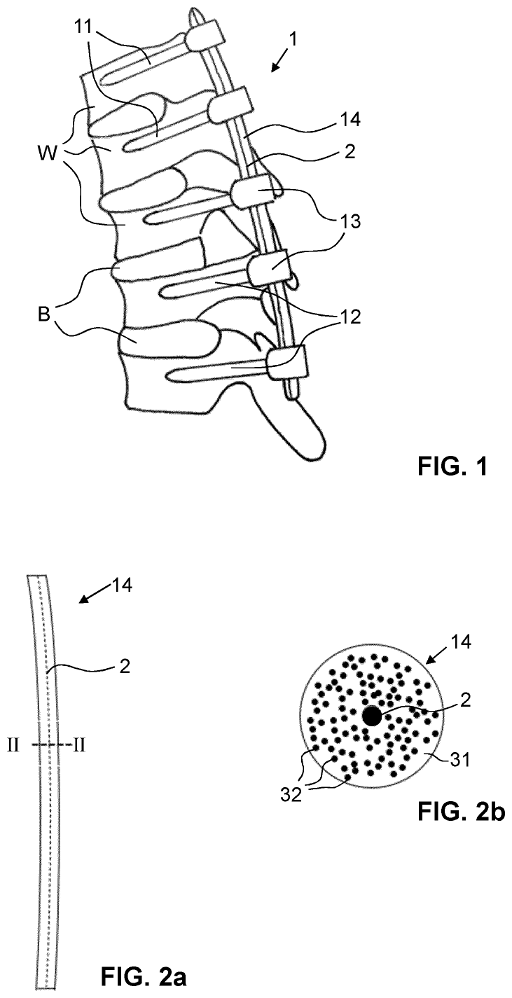

In FIGS. 1 to 9, various embodiments of medical bone implants according to the disclosure, as well as various preferred modes of manufacturing of such implants, are illustrated. Elements of the different embodiments which have an equal or similar effect are designated with the same reference numerals in the following.

FIG. 1 illustrates a dynamic pedicle fixation system 1 which is anchored in several vertebral bodies W of the spine of a patient. The vertebral bodies W are separated from each other by spinal discs B. The pedicle fixation system 1, which serves for the stabilization of the bone region shown, forms as an entirety a bone implant and comprises several pedicle screws 11, which each are screwed into a screw shaft 12 into the pedicle of a vertebral body W. Each screw is attached by means of an advantageously polyaxially adjustable tulip head 13 on a continuous connecting rod 14. The pedicle screws 11, the tulip heads 13 and the connecting rod 14 each form a bone implant of their own, due to their arrangement in the interior of the body and due to their (direct or indirect) attachment to the spine.

Other embodiments of the fixation system shown in FIG. 1 are also conceivable. Thereby, the number of pedicle screws 11 can be varied in the sense that for example instead of the five pedicle screws illustrated, only two pedicle screws are applied. Likewise, the number of pedicle screws can also exceed five. Furthermore, the pedicle system, as it normally is the case, can comprise two parallel rows of pedicle screws, which are each connected to one connecting rod.

In the interior of the slightly bent connecting rod 14, one or more sensors are arranged. In the present case, the sensor is formed by a micro wire 2, which extends in a longitudinal direction continuously along the main longitudinal center line of the connecting rod 14. Passive sensors, e.g. also in the form of a micro wire, can also be embedded in the respective pedicle screws 11.

The micro wire 2 is formed of a ferromagnetic material, which comprises an inverted magnetostriction. Upon change of the forces and/or temperature acting on the connecting rod 14 and thus on the micro wire 2, the micro wire 2 e.g. is marginally deformed, compressed, twisted and/or stretched. Due to the magnetoelastic effect, the magnetic permeability of the material of the micro wire 2 thereby changes, which is detectable and measurable by means of a reading device 5 (see FIG. 11).

FIG. 2a illustrates the connecting rod 14 of the pedicle fixation system of FIG. 1 alone from a lateral view. The slight curvature of the connecting rod 14 is recognizable. The dashed line illustrates the position of the micro wire 2, which extends directly on the main longitudinal center line of the implant and over its entire length.

In the cross-sectional view of FIG. 2b, the interior structure of the same connecting rod 14 is illustrated schematically. The implant body is formed by a plastic matrix 31, in which a plurality of carbon fibres 32 is embedded. The carbon fibres 32 extend parallel to each other along the main longitudinal center line of the connecting rod 14. This imparts the connecting rod 14 with an especially good structural stability. The micro wire 2 extends centrally in the middle of the connecting rod 14.

Thermoplastics are used as a plastic matrix 31, preferably so-called high temperature thermoplastics of the families of polyaryletherketones, polyimides, or polysulfones. The fibre length of the carbon fibres is at least 1 mm, however, preferably a length which corresponds to the complete length of the main extension direction of the connecting rod 14.

The fibre volume content is in a range of from 20 to 80%, preferably between a range of 35-70%, especially preferred between a range of 45-60%. The connecting rod 14 illustrated in FIG. 4b comprises a circular cross-section. Other cross-sectional forms, such as rectangular or hexagonal, are also conceivable.

The diameter of the connecting rod 14 illustrated in FIGS. 2a and 2b, is preferably in a range of 2-10 mm, preferably in a range of 4-6 mm. The geometric implementation in the longitudinal direction of the connecting rod 14 can be straight or curved or a combination of both, in that a section of the longitudinal direction is straight and the adjoining section is curved. A geometric implementation in the longitudinal direction, in which one section in the longitudinal direction is curved in a convex manner, and the adjoining section is curved in a concave manner, is also conceivable. The radius of curvature of the curved embodiments is preferably in a range of 50-600 mm. Other radii of curvature are also conceivable. The length of the connecting rod 14 is preferably in a range of 50-500 mm. Other lengths are also conceivable.

The sensor 2 used in the connecting rod 14 of FIGS. 2a and 2b is a passive sensor, which especially changes its specific material characteristics under the influence of thermal loads, which are then recorded in a wireless manner and transmitted from the interior of the body to the exterior of the body. Preferably, a magnetoelastic micro wire is used for this purpose, which comprises a ferromagnetic alloy as well as an inverted magnetostriction, such that the micro wire 2 changes its magnetic characteristics under the thermal load. The change of the magnetic characteristics can especially be recorded and measured by means of the magnetic permeability. The known physical phenomena therein are predominantly the Villari-effect and preferably the Barkhausen-jump. The person skilled in the art knows how the at least one sensor, i.e. in this case the micro wire 2, must be designed, and how the reading must be performed in order for the measurement to be based on the Barkhausen-effect. The person skilled in the art obtains such suggestions for example from WO 2007/116218 A1 and EP 3 150 998 B1.

Therein, embodiments of the micro wire 2 are preferably used, which have a diameter in the range of 10 μm to 250 μm and preferably extend over the entire length of the implant, either completely continuously or in pieces in adjoining sections, whereby the entire length of the component of the connecting rod 14 can be completely controlled and monitored. An especially preferred embodiment of such passive sensors are those which comprise a temperature stability of up to at least 450° C. and a have a precision in the temperature recording of at least ±0.1 K. The preferred positioning of the micro wire 2 in the connecting rod 14 is central, however, also a non-central positioning is conceivable. With the embodiment of this micro wire 2 in the connecting rod 14, it is also possible, prior to implantation, to record and transmit in a wireless manner the temperatures during the manufacturing process and/or storage of the connecting rod 14.

The number of sensors in the embodiment of FIG. 2b is one. However, several sensors of the same type can be integrated in the connecting rod 14.

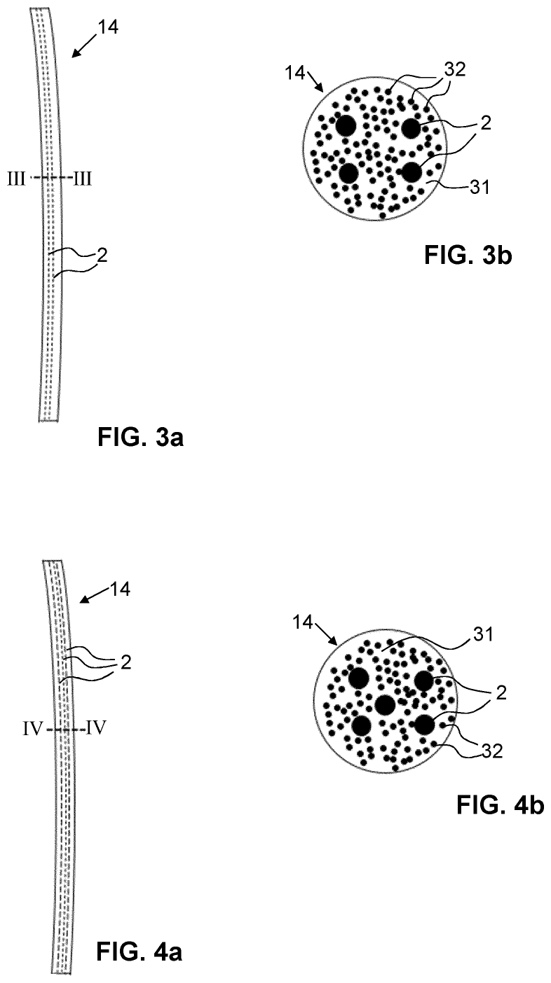

The embodiment illustrated in FIGS. 3a and 3b differs from that of FIGS. 2a and 2b in that several, specifically four micro wires 2, which run parallel to each other along the main longitudinal center line of the connecting rod 14 are provided in the connecting rod 14. All four micro wires 2, however, are arranged at a distance from the main longitudinal center line, i.e. they run in a de-centralized manner in the connecting rod 14. Here, the micro wires 2 serve to measure mechanical loads, e.g. tension, pressure, deflection and/or torsion. Of course, in other embodiments, it is also possible that more than four or less than four micro wires 2 are integrated in the connecting rod 14. A central positioning of a micro wire 2 is also conceivable, especially if it shall serve to record and measure tensile and compressive forces.

Thus, in case of a central positioning of the micro wire 2, as is the case for example in the embodiment of FIGS. 2a and 2b, tensile and compressive forces are measurable. However, in case of a de-centralized positioning of the micro wire 2, as for example in the embodiments of FIGS. 3a and 3b, additionally or alternatively bending- and torsional forces can be recorded.

The embodiment illustrated in FIGS. 4a and 4b differs from that of FIGS. 2a, 2b and 3a, 3b, in that here five micro wires 2 are provided, which extend parallel to each other within the connecting rod 14, wherein one of them is arranged approximately centrally in the connecting rod 14, and the others at a distance thereto. The four micro wires 2 which are arranged in a de-centralized manner, serve to measure mechanical loads, such as e.g. tension, pressure, deflection and/or torsion. The micro wire 2 which is arranged in an approximately centralized manner, however, serves to measure thermal loads. Of course, also other arrangements and a different number of micro wires 2 is possible.

The passive sensors provided in the embodiments of FIGS. 2a to 4b must not necessarily be in the form of micro wires, but can also have other forms.

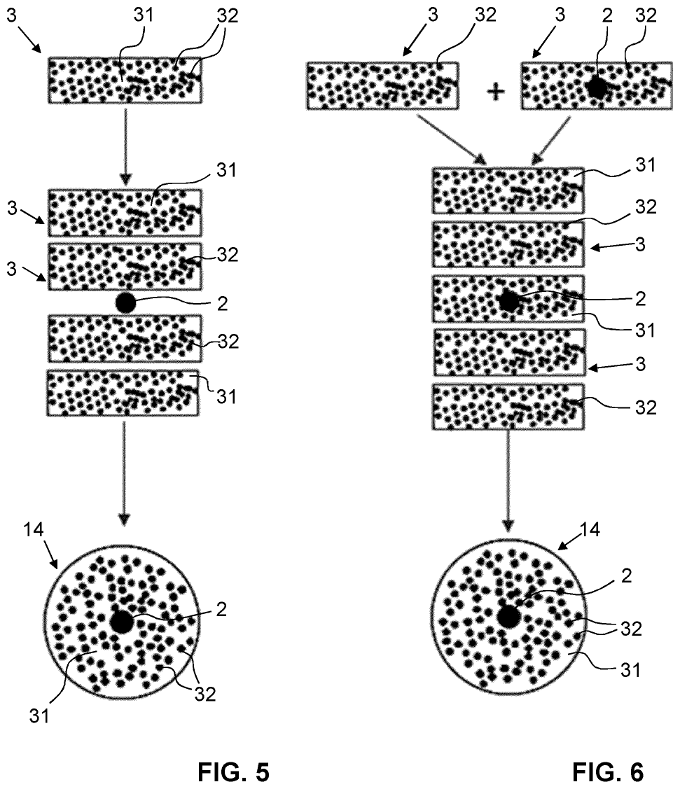

FIGS. 5 and 6 in each case represent a flow diagram which visualizes the manufacture of an implant according to the disclosure, from the raw material to the final component. The manufacture of the connecting rod 14 illustrated in FIGS. 2a and 2b serves as an example. Therein, two variants of the manufacture of the connecting rod 14 exist, which can be equally preferred.

In the first variant, which is illustrated in FIG. 5, unidirectional fibre-reinforced propregs 3 are used in this example, which in each case comprise carbon fibres 32 which are embedded in a plastic matrix 31. Likewise, tissue prepregs can also be used. The unidirectional fibre-reinforced prepregs 3 used in this first variant, are in each case geometrically laminar with a preferred width of 1-50 mm and a preferred thickness of 0.1-0.5 mm. Other dimensions in terms of width and thickness are also possible. Other geometric embodiments, such as for example circular, are also possible. The prepregs 3 are usually longitudinally fitted, wherein the length preferably at least corresponds to the length of the connecting rod 14. For fibres, preferably carbon fibres 32 are used, which comprise a fibre length of at least 1 mm, however, preferably a length which corresponds to the complete length of the main direction of extension of the connecting rod 14.

The fibre volume content is in a range of 20 to 80%, preferably in a range of 35-70%, especially preferably in a range of 45-60%. As a material for the plastic matrix 31, thermoplastics are used, especially preferably so-called high-temperature thermoplastics of the families of the polyaryletherketones, polyimides and polysulfones.

The prepregs 3 are arranged in a second step to preforms in a geometrically precise manner, or stacked, respectively, and in between, the micro wire 2 is positioned in a centralized manner. The arrangement of the prepregs 3 can be carried out in a manual or automatic manner, for example by means of a tape laying process or by means of a 3D-printer. The positioning of the sensor, in this case the micro wire 2, can be carried out in a manual or automatic manner, for example with a 3D-printer. The precision of the arrangement of the prepregs 3 and of the sensor can be improved by means of pressure and temperature in the sense that the prepregs 3 are pre-welded to each other and to the micro wire 2, and subsequently cooled.

In a third step, the preform for example is heated in a pressure-grouting tool to a processing temperature above the melting point of the plastic matrix 31, and pressure-grouted onto the end contour of the connecting rod 14 and cooled, wherein here the integrated micro wire 2 can already transmit in a wireless manner for the purpose of recording the manufacturing data relating to the pressure and the temperature of the connecting rod 14.

In the second variant, which is illustrated in FIG. 6, in this example two different types of unidirectional fibre-reinforced prepregs 3 are used as starting materials. The composition of the one type of prepregs 3 is analogous to that of the prepreg 3 used in the first variant. In the present second variant, however, first a prepreg 3 is used, which has the same composition as the other prepregs 3, however, additionally comprises a sensor in the form of a micro wire 2 embedded in a centralized manner. The lengths, thicknesses and geometries of the prepregs correspond to those of the first variant. The length of the micro wire 2 corresponds to at least the entire length of the connecting rod 14.

The prepregs 3 are, also in this second variant, arranged in a geometrically precise manner to preforms, or stacked, respectively, wherein now, however, the prepreg 3 is positioned in a centralized manner with the micro wire 2 in between. The arrangement of the prepregs 3 can be carried out manually or automatically, for example by means of a tape laying process or by means of a 3D-printer.

In a third step, here too, the preform for example is heated in a pressure-grouting tool to a processing temperature above the melting point of the plastic matrix 31, or of the prepreg 3, respectively, and pressure-grouted onto the end contour of the connecting rod 14 and cooled, wherein here too, the integrated micro wire 2 can already transmit in a wireless manner for the purpose of recording the manufacturing data relating to the pressure and the temperature of the connecting rod 14.

The variants shown and described in FIGS. 5 and 6 for the manufacture of the connecting rod 14 with integrated sensors are preferably manufacturing processes close to the end contour, i.e. implants which are manufactured in that manner usually only require a marginal post-manufacturing with respect to form and dimensions, during which the positioning of the sensors remains unaltered.

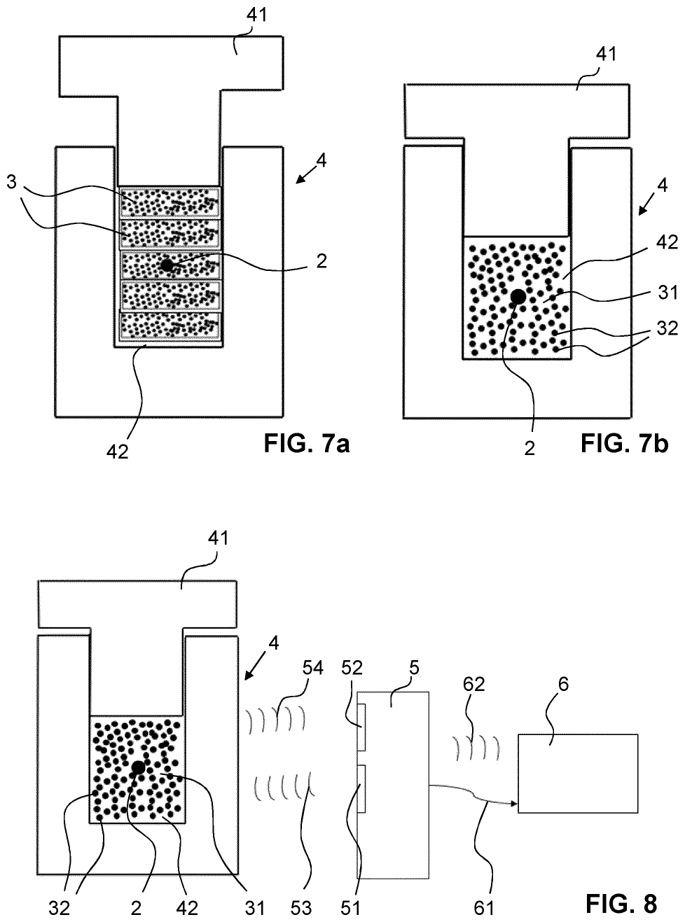

FIGS. 7a and 7b illustrate schematically a preferred manufacturing process of a connecting rod 14 according to the present disclosure with an integrated micro wire 2.

Therein, a molding tool 4 is used, which is for example manufactured of steel or of another material, and which comprises two molded parts. One of the two molded parts forms a stamp 41, and together the molded parts form a cavity 42 with a quadratic cross-section. According to the second variant illustrated in FIG. 6, the prepregs 3 are arranged to preforms at room temperature or at a temperature far below the melting point of the plastic matrix 31 in a geometrically precise manner or stacked, respectively, in the cavity 42. The prepreg 3 with the micro wire 2 is positioned in the cavity 42 in a centralized manner with respect to the other prepregs 3.

Subsequently, the preform is heated with the pressure-grouting tool 4 to a processing temperature above the melting point of the plastic matrix 31, or of the prepreg 3, respectively, and pressure-grouted onto the end contour of the connecting rod 14 by closing the two molded parts and subsequently cooled. The integrated micro wire 2 can already measure and, for the purpose of recording, transmit in a wireless manner, manufacturing data during the pressure-grouting process.

FIG. 8 illustrates a schematic illustration of the data recording and—transmittal of a preferred implant according to the disclosure during its manufacturing process. This is explained in an exemplary manner by means of the manufacturing step illustrated in FIG. 7b. The monitoring system comprises, besides the passive sensor, which is present in this case as a micro wire 2, a reading device 5 with a transmitter 51 and a receiver 52, as well as a computing unit 6. The reading device 5 is arranged in a spaced manner from the pressure-grouting tool 4 and does not comprise any cable connection associated therewith.

During the pressure-grouting process by means of the pressure-grouting tool 4, the transmitter 51 now generates, in a wireless manner, an electromagnetic excitation 53, which also covers the micro wire 2 arranged inside the connecting rod 14. Due to this excitation 53, a signal 54 is sent back from the micro wire 2, which is recorded in a for example oscillographic manner by the reading device 5 via the receiver 52. Due to the magnetoelasticity of the micro wire 2, the signal 54 therein is dependent on the pressure and the temperature, to which the connecting rod 14 and thus the micro wire 2 are subjected during the pressure-grouting process. Accordingly, based on the signal 54 received from the reading device 5, data with respect to pressure and temperature of the connecting rod 14 are collected.

These collected data are then transmitted by means of a cable transmission 61 or a wireless transmission 62 to the computing unit 6 for the purpose of subsequent analysis and recording.

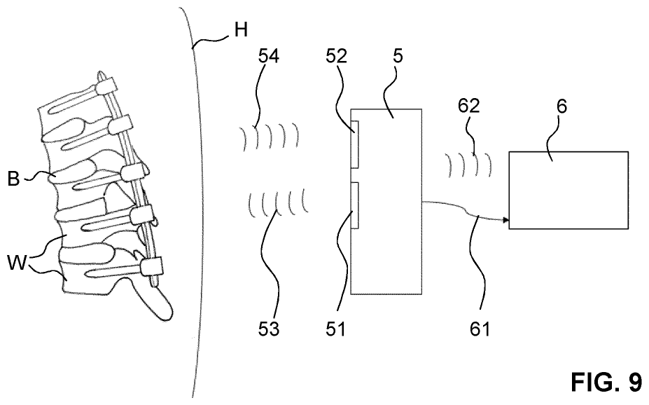

In FIG. 9, a schematic illustration of the data collection and -transmission of an implant according to the disclosure is shown in an implanted state. Here, the data collection and -transmission serve to monitor the healing process. The monitoring system is shown here in combination with the pedicle fixation system 1 illustrated in FIG. 1, in which a passive magnetoelastic sensor in the form of a micro wire 2 is integrated in the connecting rod 14. Besides the micro wire 2, the monitoring system here also comprises a reading device 5 with a transmitter 51 and a receiver 52, as well as a computing device 6. The reading device 5 and the computing device 6 are arranged outside of the body and are separated from the pedicle fixation system 1 by the skin H.

The transmitter 51, in a wireless manner generates an electromagnetic excitation 53, which among others also affects the micro wire 2 present in the pedicle fixation system 1. Due to the excitation 53, the micro wire 2 emits a signal 54 back through the skin H, which is detected by the reading device 5 via the receiver 52. The signal 54 is, due to the magnetoelasticity of the micro wire 2, dependent on the mechanical loads and the temperature, which the connecting rod 14 and thus the micro wire 2 are subjected to. Accordingly, the reading device 5 can determine data relating to the mechanical load and the temperature of the connecting rod 14, based on the signal 54 received.

The data thereby determined are then transmitted by means of a cable transmission 61 or a wireless transmission 62 to the computing unit 6 for subsequent analysis. Based on the measured data, the physician or the medical personnel can for example adjust or terminate the treatment.

1. A bone implant for stabilizing a bone region, comprising:
an implant body manufactured from a thermoplastic composite material configured for anchorage in or on a human or animal bone for the purpose of stabilizing the bone region, and at least one sensor for measuring one or more parameters of the healing process and/or of a condition of the implant,
characterized in that the at least one sensor is a passive magnetoelastic sensor.

2. The bone implant according to claim 1, wherein the at least one sensor exploits the Barkhausen-effect for measuring the one or more parameters.

3. The bone implant according to claim 1, wherein the at least one sensor is a micro wire with a diameter of preferably 10 μm to 250 μm.

4. The bone implant according to claim 1, wherein the at least one sensor is arranged at least partially or entirely in the interior of the implant body.

5. The bone implant according to claim 1, wherein the implant comprises a main longitudinal center line, and wherein the at least one sensor extends essentially along the entire longitudinal extension of the main longitudinal center line.

6. The bone implant according to claim 5, wherein the implant comprises a plurality of sensors arranged in a regularly spaced manner along the main longitudinal center line.

7. The bone implant according to claim 1, wherein the implant comprises a plurality of sensors, that extend parallel to each other.

8. The bone implant according to claim 1, further being a spine implant.

9. The bone implant according to claim 1, further being a trauma implant.

10. The bone implant according to claim 1, wherein the one or more parameters includes mechanical loads.

11. The bone implant according to claim 1, wherein the at least one sensor has a temperature stability of up to at least 450° C.

12. A method for monitoring the condition of an implant, wherein the implant comprises at least one passive magnetoelastic sensor for measuring one or more parameters of the condition of the implant, wherein the at least one sensor for monitoring the condition of the implant is used during its manufacturing process and/or during its storage.

13. The method according to claim 12, wherein the condition of the implant is determined by the at least one sensor immediately after its implantation into a human or animal body.

14. The method according to claim 12, wherein the at least one sensor monitors the healing process following the implantation of the implant into a human or animal body.

15. A method for manufacturing the implant, of claim 1, comprising providing a plurality of unidirectional prepregs,
wherein the unidirectional prepregs are pressure-grouted to each other under pressure and heat, and
wherein the at least one sensor is arranged between the prepregs or is embedded in one of the prepregs during the pressure-grouting process.

16. The bone implant according to claim 3, wherein the micro wire has a diameter of 10 μm to 250 μm.

17. The bone implant according to claim 8, wherein the spine implant is selected from a group consisting of a vertebral body replacement, a spinal disc replacement, a dowel, a screw-anchor, a fixation plate, a pedicle screw and a connecting rod of a pedicle system.

18. The bone implant according to claim 9, wherein the trauma implant is selected from a group consisting of a screw-anchor, a dowel, a bone plate, and a bone screw.

19. The bone implant according to claim 10, wherein the one or more mechanical parameters is selected from a group consisting of compressive forces, bending forces, and a temperature.
